(19) United States
(12) Patent Application Publication
(10) Pub. No.: US 2017/0274069 A1
(43) Pub. Date: Sep. 28, 2017

(54) METHOD FOR THE CANCER TREATMENT AND PREVENTION OF METASTATIC DISEASE

(71) Applicants: Anahit Ghochikyan, HUNTINGTN BCH, CA (US); Ravshan I Ataullakhanov, Moscow (RU); Michael G Agadjanyan, Huntington Beach, CA (US); Alexei V Pichugin, Moscow (RU); Alexander V Bagaev, Moscow (RU)

(72) Inventors: Anahit Ghochikyan, HUNTINGTN BCH, CA (US); Ravshan I Ataullakhanov, Moscow (RU); Michael G Agadjanyan, Huntington Beach, CA (US); Alexei V Pichugin, Moscow (RU); Alexander V Bagaev, Moscow (RU)

(21) Appl. No.: 15/521,855

(22) PCT Filed: Oct. 26, 2015
(86) PCT No.: PCT/US15/57427

§ 371 Date: Apr. 25, 2017

Related U.S. Application Data
(60) Provisional application No. 62/122,810, filed on Oct. 31, 2014.

(51) Int. Cl. A61K 39/39; A61K 39/395; C07K 16/30; C07K 16/28; C12N 5/0783; A61K 35/15; A61K 35/17; C12N 5/078; C12N 5/0786; C12N 5/0784; A61K 39/00; C07K 16/32

(52) U.S. Cl. CPC A61K 39/39; A61K 39/0011; A61K 39/39558; C07K 16/30; C07K 16/2887; C07K 16/32; C07K 16/2863; A61K 35/15; A61K 35/17; C12N 5/0648; C12N 5/0645; C12N 5/0639; C12N 5/0646; A61K 2039/55583; A61K 2039/57; C07K 2317/732; A61K 2039/5158; C12N 2501/05

(57) ABSTRACT

The disclosure provides a method of new use of an immunomodulatory drug comprising a water-soluble acidic peptidoglycan from sprouts of the plant Solanum tuberosum (WSPG) for prevention and treatment of metastatic tumor growth, comprising an application of a therapeutically effective dose of a said immunomodulatory drug or a pharmaceutical composition of a said immunomodulatory drug with a pharmaceutically acceptable carrier or excipient, or a pharmaceutically effective amount of mammalian immune cells treated with said immunomodulatory drug WSPG in vitro/ex vivo to a patient in need thereof.

### RELATED APPLICATION DATA

This application claims the benefit of U.S. 62/122,810, filed 31 Oct. 2014.

### TECHNICAL FIELD

The invention relates to the methods for tumor immunotherapy, in particular to a method for prevention and treatment of metastatic tumor growth by activating dendritic cells, macrophages and NK cells with a plant-derived immunostimulant.

### BACKGROUND

Cancer is the second leading cause of death in the world and leading cause in some developed countries. Every year about 7 million people are dying of cancer worldwide. Although, primary treatments of cancer (surgery, radiation therapy, and chemotherapy) are beneficial and lead to increased disease free and overall survival, there is a continuous relapse rate that leads to a substantial proportion of cancer patients developing recurrent and/or metastatic disease. One of the most promising strategies for fighting the development of metastatic disease is the stimulation of patient's immune system to “seek out and destroy” disseminated tumor cells. However, currently, no one has any doubt that cancer is associated with suppression of the immune system, in particular, of the immune surveillance against oncogenic viruses or abnormal cells. Therefore, integrated use of immunotherapy with conventional methods of cancer treatment can significantly increase the effectiveness of treatment.

It is known that activated macrophages are able to recognize and kill tumor cells, hence the activation of macrophages can play an important role in development of novel immunotherapeutic approaches for the treatment of cancer (Klimp et al. Crit Rev Oncol Hematol. 44:143-61, 2002). The cytotoxic functions of macrophages could be stimulated by TLR-ligands such as LPS or cytokines such as IFNγ and GM-CSF. Although several clinical trials based on monocyte or macrophage activation have been conducted (Clinicaltrials.gov, NCT00631631), this approach is restricted by (1) the limited number of drugs activating macrophages and approved for use in humans, and (2) the fact that macrophages in tumor environment lose the ability to fight the tumor and on the contrary, become “tumor nurses” (Quatromoni et al. Am J Transl Res, 4:376-89 2012).

Thus, the search for drugs that are approved for human use and are capable of activating macrophages, triggering antitumor activity, including tumor associated macrophages, is relevant.

Another important population of immune effector cells that have a unique ability to directly lyse transformed or virus-infected cells are natural killer (NK) cells. More than 40 years after the discovery of NK cells, they attract attention as means of anticancer therapy. Their name comes from the innate ability of these cells to kill tumor cells. Following the discovery that interleukin-2 (IL-2) strongly enhances the antitumor activity of NK cells, there have been numerous attempts to treat cancer patients by administering IL-2 and other cytokines (IL-12, IL-15, IL-18, IL-21, IFN-γ) into the patient's body, but they have not been very successful because of the high toxicity of these drugs (Langers et al. Biologics 6:73-82, 2012). There are numerous examples of per os administration of various plant-derived drugs, which are believed to be able to activate antitumor NK cells, however a critical overview of the literature (Ramberg et al. Nutr J 9:54, 2010) shows that in most cases the effect of these drugs on NK cells has not been proved. Therefore, there is a need to find safer substances, which can increase the antitumor activity of NK cells, and which can be used in humans, using well-controlled administration routes such as intravenous or intramuscular injection.

For over a decade, dendritic cells cultured ex vivo, are used as a cell therapy for cancer. Mononuclear cells or purified populations of monocytes isolated from the patient's peripheral blood are incubated for different periods of time with the addition of various cytokines in order to differentiate them into the dendritic cells (Ueno et al Immunol Rev 234:199-212, 2010). Often the lysate of tumor cells isolated from the same patient is added to load dendritic cells with antigens for the induction of tumor-specific T cell responses (Van Brussel et al. Mediators Inflamm 2012: Article ID 690643, 2012).

Recent studies documented that besides the orchestration of innate and adaptive immune responses some subsets of dendritic cells can also function as direct cytotoxic effectors against tumors, therefore may have important implications in tumor immunotherapy (Larmonier et al. Cancer Immunol Immunother 59:1-11, 2010). DC endowed with cytotoxic activity have commonly been referred to as “killer DC” (KDC). Different subsets of KDC have been identified in mouse (Chan et al. Nat Med 12:207-13, 2006; Yang et al. Cell Immunol 179:84-95, 1997), rat (Trinite et al. J Immunol 165:4202-8, 2000) and human (Manna et al. J Leukoc Biol 72:312-20, 2002). In these papers it was shown that the cytotoxic activity of KDC can be also induced upon activation with specific stimuli such as TLR4 ligand LPS, the TLR2 agonist Pam3Cys-SK4, or IFN-γ. However, most of these stimuli can not be used directly in human body due to high toxicity. Therefore, there is a need for safe and effective pharmaceuticals for enhancing the anti-tumor cytotoxic activity of dendritic cells.

Later, a small population of natural killer dendritic cells have been described in human peripheral blood (Lee J-M, 2013 US 2014/0011230 A1) and the anti-tumor immunotherapeutic approach has been developed based on ex vivo expansion of these rare populations of dendritic cells and further administration into the patient (Lee J-M, 2013 US 2014/0010793 A1).

### DETAILED DESCRIPTION OF THE INVENTION

The present invention involves the method for new use of a water-soluble acidic peptidoglycan from sprouts of the plant Solanum tuberosum (WSPG) for prevention and treatment of metastatic tumor growth. WSPG is a compound according to patent RU 2195308, patent application RU 2013151824 and registered in Russian Federation under No. 001919/02-2002. In one embodiment WSPG as an activator of several types of anti-tumor cells of immune system, such as dendritic cells, macrophages, NK cells, is formulated in a pharmaceutically acceptable carrier, excipient and/or diluent and administered to the patient directly.

In another embodiment peripheral blood mononuclear cells or dendritic cells or macrophages collected from blood of the patients are activated with WSPG in vitro/ex vivo and activated cells are returned to the patient in need thereof.

Present invention is based on our studies demonstrating that acting through the TLR-4 receptor, WSPG (application RU 2013151824) triggers the antitumor activity of immune system via activation of dendritic cells. WSPG increases the expression of CD80 and CD86 costimulatory molecules, HLA-DR molecules, production of IL-12, IL-6, MCP-1, TNF-α, cytokines, production of NO by macrophages.

Antitumor effect of DC, activated with WSPG is not limited to one type of tumor, it was shown for all types of tumors studied in this paper.

Apart from their own anti-tumor properties WSPG activated DC, also acquire the ability to enhance antitumor cytotoxic properties of NK cells of mouse and human.

Since the discovery of the Toll-like receptor (TLR) in human cells in 1997 (Medzhitov et al. Cell, 1997 91:295-8), TLR ligands have periodically been investigated for the treatment of cancer and inflammatory diseases (Basith et al. Expert Opin Ther Pat 21:927-44, 2011) with mixed results, likely due to their intrinsic toxicities and the context in which they have been used. WSPG is a highly effective and non-toxic immunomodulator capable to activate immune cells to fight metastatic cancer. WSPG simultaneously activates several types of antitumor immune cells, particularly WSPG converts DC to tumor killer cells, causes the production of cytokines by dendritic cells, activating NK cells which enhances their cytotoxic activity against tumor; also under influence of WSPG occurs reprogramming of macrophages into tumor killers, even tumor-associated macrophages are transformed into macrophages that actively kill various tumors.

In one embodiment of the invention, the ability of chemotherapy to induce death of cancer cells is utilized to stimulate immune responses directed to tumor antigens released by damaged tumor cells (Zhang et al. Cancer immunology, Immunotherapy CII62:1061-1071, 2013; Kang et al. Cancer Research 73:2493-2504, 2013). Said immune response is amplified by administration of WSPG systemically in order to endow a state of activation in innate immune system cells, said innate immune system cells then act to prime the immune response to released cancer antigens so as to induce protective immunity. Protective immunity in cancer is typically associated at the level of antigen presentation with production of Th1 and Th17 cytokines such as IL-12, IL-15, IL-17, IL-17F and IL-33.

In one embodiment of the invention disclosed is a method of augmenting anti-tumor immune responses in a cancer patient, said method comprising the steps of: a) selecting a cancer patient; b) assessing immune response of said cancer patient; c) administering a composition containing an effective amount of WSPG to augment said immune response; and d) adjusting dosage of said WSPG based on modulation of immune response. Said immune response assessment is performed by assessing function of immune cells, said immune cells selected from a group comprising of: a) B cells; b) T cells; c) innate lymphoid cells; d) natural killer cells; e) natural killer T cells; f) gamma delta T cells; g) macrophages; h) monocytes; i) dendritic cells; j) neutrophils; and k) myeloid derived suppressor cells. B cells may be either CD5+ B cells or CD5− B cells, furthermore, said B cells may be naïve B cells or memory B cells. In the case of T cells, said T cells are either CD4+ or CD8+ T cells, with selection made between memory and naïve T cells. In the situation of cancer, desired immune stimulation is associated with production of cytokines or biological mediators that inhibit cancer, or cancer associated angiogenesis. Assessment of said modulators is performed by ELISA, ELISPOT, Luminex bead assay, or other means known in the art to quantify production of biological mediators such as cytokines. Said cytokines of particular relevance to cancer include production of Th1 or Th17 cytokines, which have been shown to associate with anticancer activity. Additionally, reducing expression of immune suppressive cytokines such as IL-10 and TGF-beta is one desired outcome of the invention. In one embodiment of the invention a cancer patient is selected based on low immune response, treated with a therapeutic dose of WSPG systemically, and immune response levels are assessed. Based on manipulation of immune response dosing schedule of WSPG is modified. It is desirable in cancer patients to possess a normal or heightened NK cytotoxic activity. Means of assessing NK cytotoxicity activity are known in the art and include the K-562 assay. Other cytotoxic components of the immune system that are modulated by WSPG administration include monocytes, macrophages, and dendritic cells. In addition, cytotoxicity of cells such as CD8 cytotoxic T cells is also desired in cancer patients. The cytotoxicity of immune cells is in many cases regulated by the T helper cell. In situations of anticancer immunity T helper cells such as Th1 or Th17 produce cytokines such as IFN-gamma that endow cytotoxic activity on NK cells or macrophages. Specifically, the production of these cytokines is associated with successful anticancer immune responses. In one embodiment the use of WSPG is utilized to enhance Th1 and Th17 differentiation. Assessment of Th1 cell numbers can be performed by assessment of markers associated with Th1 cells, such markers include; a) CD4; b) CD94; c) CD119 (IFNγ R1); d) CD183 (CXCR3); e) CD186 (CXCR6); f) CD191 (CCR1); g) CD195 (CCR5); g) CD212 (IL-12Rβ1&2); h) CD254 (RANKL); i) CD278 (ICOS); j) IL-18R; k) MRP1; 1) NOTCH3; and m) TIM3. Assessment of cell surface markers is known in the art and includes utilization of flow cytometry, confocal microscopy, or mRNA assessment of surface markers.

In another embodiment of the invention, administration of WSPG is performed to suppress activity of T regulatory cells, or conversely to inhibit sensitivity of cells to the suppressive effects of T regulatory cells. Assessment of T regulatory cells is well known in the art and includes quantification of markers such as; a) CD25; b) CTLA-4; c) membrane bound TGF-beta and d) FoxP3. Other immune cells may be used for assessment of overall patient immunity and as a guide to adjusting treatment of WSPG, such cells include innate lymphoid cells, said cells are selected from a group comprising of: a) innate lymphoid cells 1; b) innate lymphoid cells 2; c) innate lymphoid cells 3 and d) lymphoid tissue inducer cells. Innate lymphoid cell 1 express T bet and respond to IL-12 by secretion of interferon gamma, however lack expression of perforin and CD56, innate lymphoid cell 2 produce IL-4 and IL-13, innate lymphoid cell 3 produce IL-17a and IL-22.

In one embodiment the use of WSPG is utilized for inducing an inflammatory response in a cancer patient, with dosage of WSPG given to patient being guided by inflammatory markers in the blood of said cancer patient. Inflammatory marker may be selected from a group comprising of: C reactive protein (CRP); IL-1; IL-6; IL-8; IL-11; IL-17; IL-21; IL-33; TNF-alpha; lipoprotein-associated phospholipase A2 (LP-PLA2); lipoprotein Lp(a); myeloperoxidase (MPO); macrophage chemotactic protein 1 (MCP-1); oxidized low-density lipoprotein (oxidized LDL), adiponectin, matrix metalloproteases (MMP), such as MMP-9,1,2; CD40; homocysteine; cardiovascular risk factor (CVRF); plasminogen activator inhibitor (PAI-1); prostaglandin (PG); tissue polypeptide antigen (TPA); von Willebrand factor (vWF); platelet aggregation; fibrinogen; Factor VII; Factor VIII; tissue factor; phosphoglucose (PGI1); endothelin; metaloproteinases; Lipoxygenase; and angiotensin.

In another embodiment the use of WSPG is disclosed as an adjuvant to tumor antigen vaccination, or to potentiate mature dendritic cells to present said tumor specific antigens. Useful tumor specific antigens. Tumor-specific or tumor associated antigen is selected from a group comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAXS; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS (CTCFL); bk) ML-IAP; bl) idiotype of b cell lymphoma or multiple myeloma; bm) EphA2; bn) EGFRvIII; bo) cyclin B1; bp) RhoC; bq) androgen receptor; br) survivin; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; ce) NR2F6; and cf) Chondroitin sulfate proteoglycan 4 (CSPG4).

In one embodiment of the invention, administration of WSPG is performed together with an antibody targeting cancer or cancer associate mediators. Of particular relevance to the practice of the invention is the use of antibodies whose therapeutic activities are associated with antibody dependent cellular cytotoxicity (ADCC). The practitioner of the invention has several antibodies well known to be useful in the treatment of cancer, these include 3F8, 8H9, Abagovomab, Abituzumab, Adecatumumab, Afutuzumab, Amatuximab, Anatumomab mafenatox, Anetumab ravtansine, Apolizumab, Arcitumomab, Ascrinvacumab, Atezolizumab, Bavituximab, Bevacizumab, Bivatuzumab mertansine, Blinatumomab, Brentuximab vedotin, Brolucizumab, Brontictuzumab, Cantuzumab mertansine, Cantuzumab ravtansine, Catumaxomab, Cetuximab, Cixutumumab, Zatuximab, Votumumab, Vorsetuzumab mafodotin, Volociximab, Vanucizumab, Vantictumab, Vandortuzumab vedotin, Urelumab, Ulocuplumab, Ublituximab, Tucotuzumab celmoleukin, Tremelimumab, TRBS07, Trastuzumab, Tigatuzumab, Ticilimumab, Tenatumomab, Tarextumab, Taplitumomab paptox, Tacatuzumab tetraxetan, Sofituzumab vedotin, Siltuximab, SGN-CD33A, SGN-CD19A, Sibrotuzumab, Robatumumab, Rituximab, Rilotumumab, Ramucirumab, Radretumab, Pritumumab, Pidilizumab, Pertuzumab Omnitarg, Lumretuzumab, Isatuximab, Ipilimumab, Enavatuzumab, Edrecolomab, Duligotumab, Clivatuzumab

In another embodiment WSPG is administered in combination with androgen ablation in order to augment immunological effects of androgen ablation. The immunological effects of androgen ablation are apparent in animal models of autoimmune disease. For example, in the non-obese diabetic (NOD) mouse, predominance of disease onset is in females, which develop diabetes spontaneously in approximately 80% incidence, whereas male incidence is approximately 20%. Interestingly, when male mice are castrated an increase in diabetes incidence occurs. Conversely, administration of testosterone in female NOD mice results in suppression of diabetes (Makino et al. Jikken Dobutsu 30:137-40, 1981). Supporting a tolerogenic role for androgens are studies in which androgenic compounds such as the drug danazol prolongs allograft survival through upregulation of T regulatory cells (Uchiyama et al. Transpl Int 25(3):357-65, 2012; Uchiyama et al. Transplant Proc 44:1067-9, 2012). Indeed T regulatory cells have been shown to possess androgen receptor and their activity is upregulated by culture with physiological levels of testosterone (Makino et al. Jikken Dobutsu 30:137-40, 1981).

It is known that androgen ablation induces an increase in T cells infiltrating the prostate, contributing to reduction of tumor mass. Specifically, it has been shown that 1-4 weeks after initiation of androgen ablation therapy there is an increase in CD4 and CD8 T cells that infiltrate the prostate, with indication that an antigen-specific expansion may be occurring as evidenced by restricted TCR Vβ gene usage (Makino et al. Jikken Dobutsu 30:137-40, 1981). Supporting a functional role for infiltrating immune cells are studies showing that elevated abundance of NK cells was associated with a lower risk of prostate cancer progression, while a high density of CD68(⁺) macrophages was related to an increased risk of biochemical recurrence (Makino et al. Jikken Dobutsu 30:137-40, 1981).

In one embodiment of the invention administration of WSPG is performed to enhance NK cell activity in patients receiving androgen ablation, so as to augment possibility of reducing risk of prostate cancer progression. Furthermore, in another embodiment of the invention, administration of WSPG is performed to reduce the tumor promoting activity of macrophages that are infiltrating the tumor. It is known that on one hand tumors possess the ability to modify macrophages into an “M2” phenotype, which promotes tumor growth and tumor associated immune suppression. On the other hand, it is known that under certain situations “M1” macrophages are associated with reduction of tumor growth. Accordingly, it is within the scope of the current invention to utilize WSPG to: a) stimulate an increase in the number of M1 macrophages entering the prostate cancer tissue; b) reduce the number of M2 macrophages that enter prostate cancer tissue; c) stimulate activity of M1 macrophages; and d) decrease activity of M2 macrophages. Alternatively WSPG may be utilized ex vivo to generate M1 polarized macrophages which may be administered in conjunction with androgen depletion.

Thus, in the present invention we propose a new use of WSPG for treatment or prevention of the metastatic tumor growth alone and in combination with other used treatments such as chemotherapy, radiation, hormone therapy, anti-angiogenic therapy, surgery, checkpoints inhibitors, adoptive cell transfer, vaccines, other immunomodulators etc. The proposed approach provides the following benefits: activation of anticancer properties simultaneously in several types of cells: monocytes/macrophages, NK cells, dendritic cells, T and B cells; the used pharmacological agent is approved for human use.

### BRIEF DESCRIPTION OF THE DRAWINGS

FIGS. 1 A and B show killing of 4T1 cell line as well as metastatic 4T1 cells by mouse splenocytes in vitro activated with WSPG (A) shows that naive splenocytes and WSPG alone did not affect the growth of 4T1 cells in vitro, whereas incubation of splenocyte/4T1 co-culture with WSPG led to complete inhibition of 4T1 cell growth. Photographs of the respective wells are presented. (B) shows that activation of splenocytes isolated from 4T1 tumor bearing mice with WSPG inhibited the growth of splenic metastatic tumor cells.

FIG. 2 shows that WSPG enhances the antitumor activity of mouse macrophages against 4T1 cells in vitro. Peritoneal macrophages from Balb/c mice were co-cultured in the wells of 96-well plate in different ratios with 4T1 tumor cells (50 cells) for 6 days in a medium (white squares) or medium, supplemented with WSPG (black dots). All samples were analyzed in triplets. The color intensity of the 4T1 tumor cell colonies is normalized to the rate in the control 4T1 cell monoculture (mean and standard deviation). P<0.01.

FIGS. 3 A and B show that normal macrophages and tumor associated macrophages, acquire the capacity to kill 4T1 tumor cells as a result of activation by WSPG. Peritoneal macrophages from tumor-free mice (low-dotted bars) and mice inoculated in the peritoneal cavity with 150000 4T1 cells (high-dotted bars), were washed out of the peritoneum cavity, and then sorted as F4/80 positive cells using FACS Aria II (BD Biosciences) in triplets. A. Sorted macrophages were seeded in the wells of 96-well plate at a density of 1×10⁵ cells/well, and cultured for 48 hours in complete medium supplemented with 10 μg/ml WSPG (2) or without the addition of WSPG (1), and then the supernatant was collected and the concentration of NO was evaluated as the stable oxidation product of NO into nitrite in Griess reaction. B. Sorted macrophages were seeded in the wells of 96-well plate at a density of 6×10³ cells/well and co-cultured with 200 4T1-GFP cells for 4 days in the presence of 10 μg/ml WSPG (2) or without it (1). The number of live 4T1-GFP cells was determined using FACS Aria II after staining with propidium iodide (2 μg/ml). White bar—monoculture of 4T1-GFP cells without the addition of macrophages, black bars—co-cultures of 4T1-GFP cells with macrophages from tumor-free mice, gray bars—co-cultures of 4T1-GFP cells with macrophages, associated with the tumor. Mean values for the three experiments are presented. Significance of differences * p<0.05, ** p<0.01.

FIG. 4A-C show killing of tumor cells by highly purified BMDC activated with WSPG. A and B show the purity of NK cells isolated from naive mouse spleen (A) and purity of bone marrow derived dendritic cells (BMDC) (B). (C) Antitumor effect of highly purified NK cells and BMDC activated by WSPG during co-cultivation with 4T1 tumor cells in vitro. 4T1 cells (200 cells per well in 96-well Plate) were co-cultured either alone or with purified NK cells (50,000 cells per well), BMDC (25,000 cells per well) or NK/BMDC in the presence of 10 μg/ml WSPG (low-dotted bars) or without it (high-dotted bars). * P<0.05.

FIG. 5A-E show the activation and proliferation of NK cells by Dendritic cells pre-activated with WSPG. (A, B, C)—analysis by flow cytometer FACS Aria II (BD Biosciences, USA). Allocation strategy of NK cells as negative for CD4 and CD8 (A) and negative for CD19, but positive for DX5 (B). Activated and proliferating NK cells were determined by increased expression of CD69 and a decrease in fluorescence CellTraceViolet (C). (D). Murine BMDC were activated with or without WSPG [incubation for 18 hours in the absence of WSPG (black squares) or in the presence of 10 μg/ml WSPG, white squares], and added in different proportions to murine splenocytes stained with CellTraceViolet (CTV). After 4 days incubation flow cytometry was performed, the percentage of activated CD69⁺, DX5⁺, CD4⁻, CD8⁻, CD19⁻ and proliferating (reduced content CellTraceViolet) NK cells was calculated. Mean values and standard deviations from three independent experiments are presented. Significance of difference (*) P<0.05. (E) Highly purified dendritic cells pre-activated with WSPG, activate the highly purified NK cells. High-dotted bars—monoculture of 5×10⁴ highly purified NK cells, low-dotted bars—the co-culture of highly purified NK cells (5×10⁴) and dendritic cells (2.5×10⁴). Mean values and standard deviations from two independent experiments are presented. Significance of difference (*) P<0.05.

FIG. 6 shows the activation of NK cells in mice injected with WSPG intravenously. BALB/c mice (female) were administered with WSPG (10 μg per mouse) through orbital sinus. After 18 hours blood mononuclear cells were taken. YAC-1 target cell line was stained with CellTraceViolet™ Proliferation Kit (Invitrogen). Target cells (T) and effector cells (E) were incubated at various ratios of E:T in round bottom 96-well plates for 4 hours. Cells were stained with propidium iodide and the percentage of killed target cells was evaluated by flow cytometer FACS AriaII. Black squares—mice received an intravenous injection of saline (control group), white squares—mice treated with intravenous injection of WSPG. Mean values with standard deviations from 4 independent experiments are presented. * P<0.05

FIGS. 7A and B show that administration of WSPG inhibits metastatic disease and prolongs the survival of 4T1 tumor-resected mice. 15×10³ 4 T1 tumor cells were injected subcutaneously in the mammary gland of female BALB/c mice. Upon reaching a diameter of 3.5-5 mm tumors were surgically removed. WSPG (10 μg in 100 μl) was injected intravenously via the retro-orbital sinus when tumor became palpable and weekly after tumor removal. (A) Three injections of WSPG significantly decreased the number of clonogenic tumor cells in the lungs of mice. Number of clonogenic 4T1 cells in the lungs of control mice (black circles, n=8) and mice treated by intravenous injection of a solution of WSPG (white triangles, n=9) was determined at day 20 post-surgery, after three injections of WSPG. Significance of differences between groups is defined by an unpaired t-test (*P<0.05). (B) Continued weekly injections with WSPG until the 69^{th} day significantly prolonged the survival of mice (***P<0.0001). Thirty one percent of mice from this group were completely cured from the disease. Survival curves were compared using the log rank test with GraphPad Prism. Black circles—control group (n=14), isotonic NaCl solution without WSPG, white triangles—experimental group (n=15), WSPG 10 ng/100 μl per mouse weekly. Significance of differences between the groups (***), defined in the test Log—rank (Mantel-Cox) is 0.001.

FIGS. 8A and B show that WSPG increases the frequency of activated NK (A) and CD8⁺ (B) cells in spleens of 4T1 tumor-resected mice. 15×10³ 4 T1 tumor cells were injected subcutaneously in the mammary gland of female BALB/c mice. Upon reaching the diameter of 3.5-5 mm the tumors were surgically removed. WSPG (10 μg in 100 μl) was injected intravenously via the retro-orbital sinus when tumor became palpable and on days 6 and 13 post-resection. On day 20 post-resection the animals were sacrificed and the activation state of NK cells and CD8⁺ T cells in the spleen was evaluated by flow cytometry. Significant differences * P<0.01.

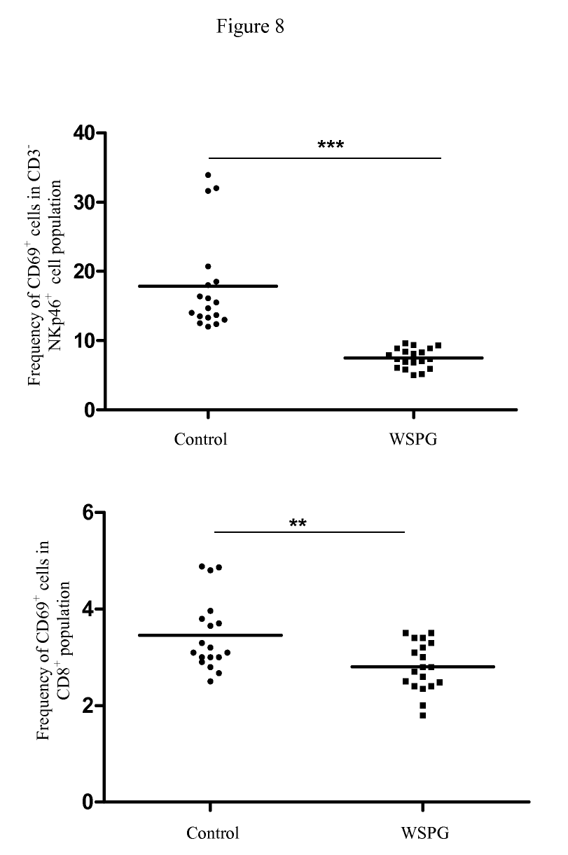

FIGS. 9A and B show that administration of BMDC pre-activated ex vivo with WSPG inhibited tumor growth (A) and decreased the number of clonogenic tumor cells in lungs (B) of tumor bearing mice in 4T1 metastatic breast cancer model. 7×10³ 4 T1 tumor cells were injected subcutaneously into the mammary gland of female BALB/c mice. 5×10⁵ BMDC pre-activated ex vivo with 30 ng WSPG were administered subcutaneously in experimental mice (n=8) three times with weekly intervals. Mice in control group (n=8) were administered with vehicle only. On day 19 mice were sacrificed and the number of clonogenic 4T1 tumor cells in lungs were quantified. (A) The volume of the tumor in cm³. Black circles—mice treated with BMDC/WSPG injection, white squares—control group. (B) The number of clonogenic metastatic cells in the lungs of mice. Low-dotted bars—mice injected of BM-DC combined with WSPG, high-dotted bars—control group. * P<0.05

FIG. 10 shows the ability of WSPG to enhance the cytolytic activity of NK cells against human tumor cells K526. Mononuclear cells from peripheral blood of healthy donors were incubated for 18 hours in the presence of WSPG (10 ng/ml). K562 target cells were stained with a set CellTraceViolet™ Proliferation Kit (Invitrogen). Effector cells (E) and target cells (T) were incubated at various ratios of E:T in round bottom 96-well plates for 4 hours. Cells were stained with propidium iodide and the percentage of killed target cells was counted on a flow cytometer FACS Aria II. Mean values and standard deviations of the results of experiments with blood cells from three donors are presented. * P<0.05

### EXAMPLES

### Example 1

Mouse splenocytes activated with WSPG in vitro are capable of killing 4T1 cell line as well as metastatic 4T1 cells

This example shows that after being activated with WSPG splenocytes from tumor-free mice as well as mice inoculated with 4T1 tumor (breast cancer) can kill 4T1 cells in vitro.

4T1 cells were seeded in the wells of 96-well plate at a density of 50 cells/well in 200 μl of complete DMEM and cultured in quadruplicates either alone or in the presence of 5×10⁴ splenocytes from naive BALB/c mice, and in the presence or absence of WSPG (10 μg/ml). Cultures were incubated for 7 days at 37° C. and 5% CO₂. For visualization and quantification, 4T1-colonies were fixed using 1% paraformaldehide, stained using 0.5% methylene blue in 50% ethanol. Digital images of each well were taken, then the integrated color density (blue channel) was calculated using ImageJ software (NIH, USA).

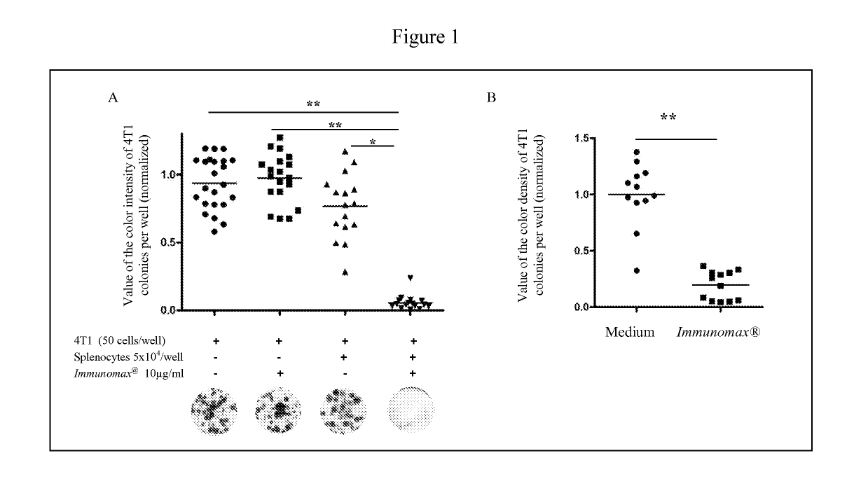

As shown in FIG. 1A adding WSPG to monoculture of 4T1 cells does not alter their viability or the rate of reproduction—the number and size of 4T1 colonies in the presence of WSPG does not differ from the number and size of colonies in monoculture of 4T1 cells without WSPG after 6-7 days of culture.

Co-culturing of 4T1 cells with 5×10⁴ tumor-free mouse splenocytes also had no effect on tumor cell growth. However, adding WSPG (10 ng/ml) into co-cultures of 4T1 cells/splenocytes, dramatically inhibited tumor cell growth.

According to the literature it is known that the development of 4T1 tumors induces large number of myeloid derived suppressor cells (MDSC) that inhibits the function of the immune effector cells (Gabrilovich et al. Nat Rev Immunol 12:253-68, 2012). Indeed, in the spleens of mice inoculated with 4T1 tumor, we observed the increasing content of the MDSC (CD11b⁺ Gr1⁺ cells). 25-30 days after subcutaneous inoculation of 7×10³ 4 T1 cells, when the primary tumor reached the size of 1 cm³, the level of MDSC in the spleens of tumor-bearing mice reached to 60-70%. It was of great interest to determine whether WSPG can activate antitumor properties of splenocytes when it is massively infiltrated by suppressor cells. FIG. 1B shows that splenocytes from tumor-bearing mice almost completely inhibited the growth of 4T1 cells in the presence of WSPG in vitro despite the presence of 70% of MDSC.

### Example 2

### WSPG Enhances Antitumor Properties of Mouse Macrophages In Vitro

In Example 1, we demonstrated that mouse splenocytes activated by WSPG inhibited the growth of 4T1 breast cancer cell line in vitro. To clarify the nature of the cells that have anti-tumor effect when activated by WSPG, we investigated isolated populations of various cell subsets of the immune system.

Tissue macrophages were obtained from the peritoneal cavity swabs of female BALB/c mice. 5 ml of PBS supplemented with 1% glucose and 1 mg/ml bovine serum albumin (BSA) was administered into the peritoneal cavity of mice (mPBS, pH 7,4). After several minutes of gentle massage frontal abdominal wall was dissected, peritoneal washings were collected with plastic Pasteur pipette, further the peritoneal cavity was washed three more times, each time introducing 5 ml of PBS. All portions of washes were combined in the centrifuge tube, cells were pelleted by centrifugation at 1200 rpm/min for 10 min, resuspended in complete culture medium at the concentration of 1×10⁶ cells/ml (RMPI-1640 supplemented with 10% fetal bovine serum, 2 mM L-glutamine, 50 mM β-mercaptoethanol and 10 μg/ml of gentamicine). Cells were incubated overnight at 37° C. and 5% CO₂. Next day, non-adherent cells were washed with warm PBS, pH 7.4, Versene solution was added to adherent macrophages and cells were kept in the refrigerator for 60 min at 4° C., and then washed off with a jet of Versene solution. Macrophages were pelleted by centrifugation at 1200 rpm/min for 10 min and resuspended in CM. Various amounts of macrophages were incubated in wells of 96-well plate (in a volume of 200 μl per well in triplets) with the 4T1 tumor cells (50 cells/well) in the presence of WSPG (10 μg/ml) or without it. After 6 days of incubation at 37° C. and 5% CO₂, The number of 4T1 colonies was quantified as described in Example 1.

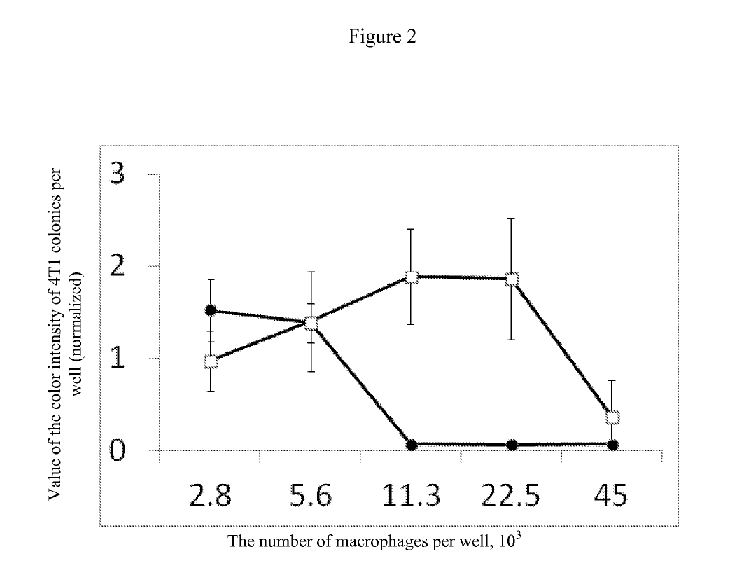

FIG. 2 shows quantitative analysis of the color intensity. It is evident that the macrophages themselves are capable of killing tumor cells at a sufficiently high amount of 45×10³ cells per well. Adding WSPG greatly enhances the antitumor activity of macrophages. 11×10³ macrophages per well activated by WSPG are sufficient for complete inhibition of the tumor growth. Thus, WSPG increased the antitumor activity of macrophages more than 4 fold.

### Example 3

### WSPG Converts Tumor-Associated Pro-Tumoral Macrophages, into Killer Macrophages

To obtain macrophages, associated with a tumor, 1.5×10⁴ 4 T1 tumor cells were injected into the peritoneal cavity of BALB/c mice. The tumor growth rate was determined by detection of MDSC in blood of injected mice (Espagnolle et al. Cancers (Basel), 6:472-90, 2014). After 7-14 days, when MDSC level in blood was above 10⁶ cells/ml the mice were sacrificed and the peritoneal macrophages were eluted as described in Example 2. In order to achieve a greater purity of the population, macrophages were stained with APC-conjugated anti-F4/80 antibodies and by FACS Aria II (BD Biocseinces).

To determine the NO producing ability of WSPG-activated macrophages, 1×10⁵ cells were seeded per well and cultured in complete medium for 48 hours with or without addition of WSPG. Then the supernatant was taken and NO production was estimated from determining the concentrations of nitrite and nitrate end products of NO oxidation using calorimetric method based on Griess reaction (Green et al. Anal Biochem 126:131-8, 1982).

To determine the antitumor activity of macrophages sorted cells (6×10³ macrophages/well) were co-cultured with 4T1-GFP cells in complete culture medium for 4 days. 4T1-GFP cells were harvested and the number of live 4T1-GFP cells was determined by flow cytometry on FACS Aria II (BD Biosciences) after staining the cells with propidium iodide.

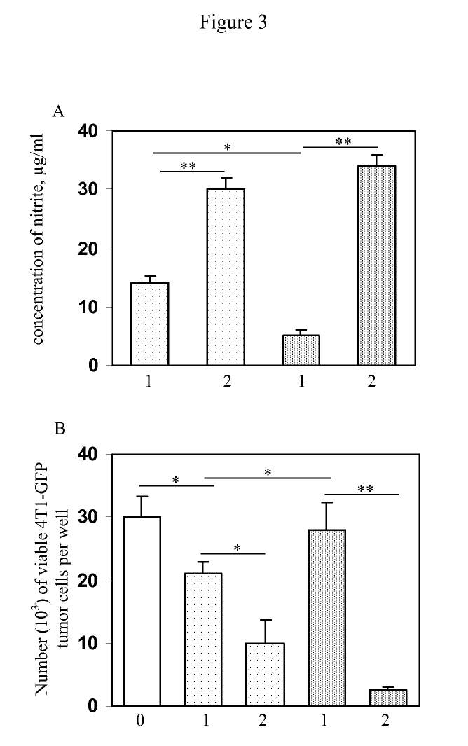

As shown in FIG. 3A the level of NO production by tumor-associated macrophages is significantly (p<0.05) reduced compared with macrophages isolated from tumor-free mice indicating their pro-tumoral properties. Importantly, incubation in the presence of WSPG significantly increased the level of NO production in both populations: in macrophages from tumor-free mice and in tumor-associated macrophages, rising it to almost the same level.

Data presented in FIG. 3B shows that macrophages isolated from tumor-free mice significantly inhibited growth of 4T1 tumor cells while the tumor-associated macrophages did not affect the growth of cells. Adding WSPG to the co-culture led to activation of antitumor properties of both populations and inhibition of the growth of 4T1 tumor cells by macrophages isolated from tumor-free mice as well as tumor-associated macrophages.

### Example 4

### Various Preparations Based on Acidic Peptidoglycan from Sprouts of Solanum tuberosum Stimulate Antitumor Activity of Mouse BMDC

We compared the antitumour properties of several preparations of the same type of water-soluble acidic peptidoglycans from sprouts of Solanum tuberosum, in particular WSPG-1 [RU 2195308], similar thereto WSPG-2 [RU 2013151824] and the pharmaceutical formulation, WSPG-3 (R No. 001919 Identification Number/02).

Mouse BMDC (4×10⁴/well) were co-cultured with 4T1-GFP tumor cells (200 cells/well) in the presence of WSPG-1, WSPG-2 or WSPG-3 (10 μg/ml). After 4 days numbers of live 4T1-GFP tumor cells were counted in the wells by flow cytometery after staining with propidium iodide. The data in Table 1 shows that stimulation of antitumor activity of BMDC is a common feature for all the investigated formulations of acidic peptidoglycan from sprouts of Solanum tuberosum.

TABLE 1
The inhibition of 4T1 tumor cells by mouse
BMDC activated by different WSPG drugs

| | Number of live | | | |
|---|---|---|---|---|
| | 4T1 cells/well | | | |
| | | Standard | Percent | |
| Sample | Mean | deviation | inhibition | Significance |
| 4T1 tumor cells | 5667 | 317 | | |
| 4T1 tumor cells + | 239 | 68 | 96% | 0.001 *** |
| BMDC + WSPG-1 | | | | |
| 4T1 tumor cells + | 66 | 2 | 99% | 0.001 *** |
| BMDC + WSPG-2 | | | | |
| 4T1 tumor cells + | 64 | 20 | 99% | 0.001 *** |
| BMDC + WSPG-3 | | | | |

### Example 5

### Sorted Dendritic Cells Stimulated with WSPG Independently Kill Tumor Cells as Well as Activate Antitumor Activity of NK Cells

In this example, we have shown a direct activating effect of WSPG on pure population of BMDC as well as NK cells, which are widely recognized as the killers of tumor cells.

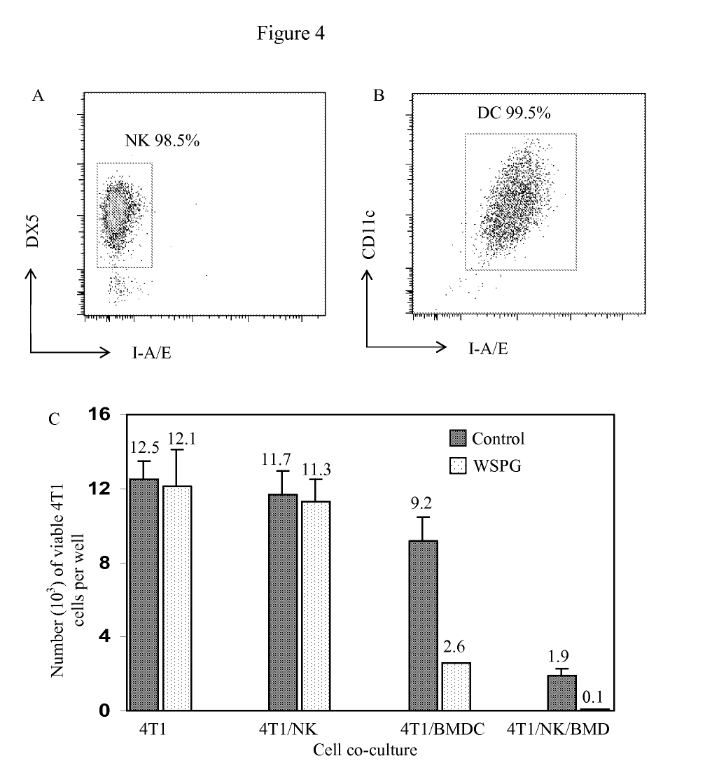

Mouse BMDC cells have been purified by cell sorter FACS Aria II to a purity of 99% by the selection of cells positive for CD11c and IA/E (FIG. 4B). Highly purified NK cells were prepared from a suspension of naive mouse splenocytes in two stages. First NK cells were enriched to a purity of 70-80% using EasySep™ Mouse NK Cell Enrichment Kit (STEMCELL Technologies Inc. Canada) according to the manufacturer's protocol, and then 98-99% purity was reached by cell sorting on BD FACS Aria II, as cells positive for DX5⁺ and negative for I-A/E (FIG. 4A).

4T1-GFP Tumor cells were cultured (at density 200 cells/well) in monoculture or in combination with highly purified NK cells and/or highly purified BMDC. As shown in FIG. 4C, highly purified NK or highly purified BMDCs individually were unable to inhibit 4T1-GFP growth. However co-cultures of these purified populations of cells exerted significant anti-4T1-GFP tumor effect (**P<0.01). Importantly, although WSPG-2 itself had no effect on 4T1-GFP growth, the addition of WSPG-2 either to the BMDCs or NK/BMDC co-cultures dramatically improved the inhibition of 4T1-GFP growth (***P<0.001). No effect was observed when WSPG-2 was added to the NK cells (FIG. 4C).

### Example 6

### Activation of NK Cell by WSPG is Mediated by Mouse Dendritic Cells

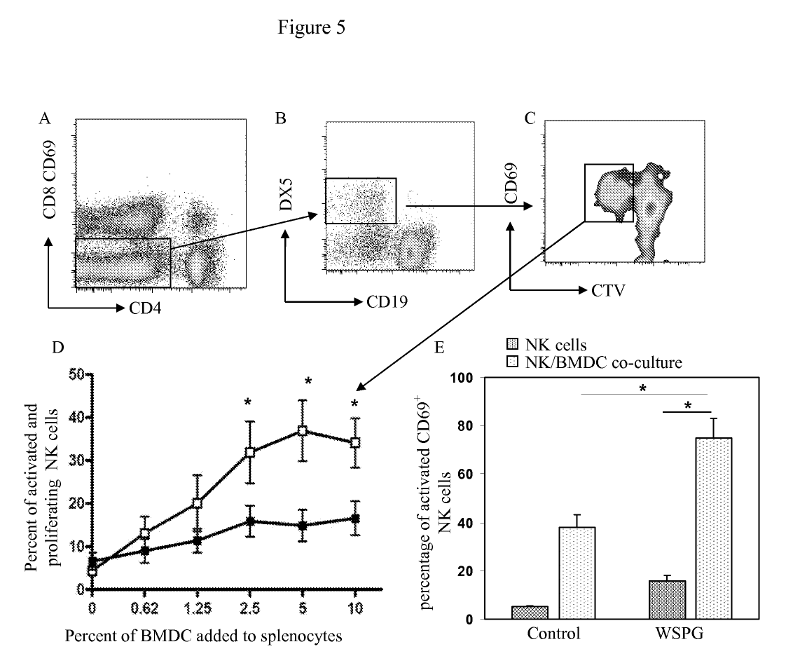

Example 5 shows that WSPG-2 does not directly activate purified splenic NK cells, but does significantly increase the capacity of BMDC to activate NK cells. Murine BMDC were pre-activated with WSPG-2 (incubation for 18 hours in the presence of 10 μg/ml) and added in different proportions to murine splenocytes stained with CellTraceViolet (CTV). After 4 days the percentage of activated (CD69⁺ DX5⁺ CD4⁻CD8⁻CD19⁻) and proliferating (reducing CTV) NK cells was determined by flow cytometry (FIG. 5 A, B, C). Data show that significantly higher (P<0.05) number of activated NK cells was detected when splenocytes were co-cultured with 2.5-10% of purified BMDC pre-activated with WSPG (FIG. 5 D).

Direct activating effect of dendritic cells previously stimulated with WSPG was confirmed using highly purified populations of NK and BMDC cells (FIG. 5E). Highly purified NK cells (5×10⁴/well) and BMDC (2.5×10⁴/well) were co-cultured for 18 hours without (control) or with WSPG and percentage of activated NK cells (CD69⁺ cells) was determined. BMDC themselves activated considerably NK cells. However, the addition of WSPG to the NK/BMDC co-cultures dramatically increased the percentage of activated NK cells and led to a 2-fold increase in the level of expression of the CD69 on the NK cells (FIG. 5E).

### Example 7

### WSPG Injected Intravenously into Mice, Enhances the Cytolytic Activity of NK Cells

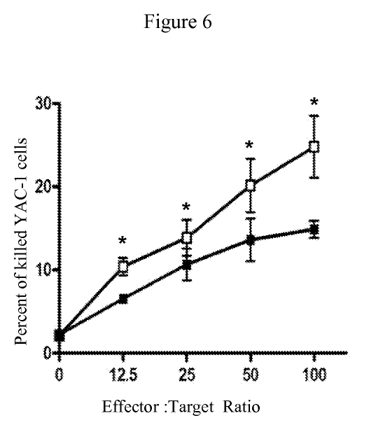

Example 6 shows the effect of WSPG on activation of mouse NK cells in vivo. BALB/c mice (n=12/group) were administered with 10 μg WSPG-2 through orbital sinus. Mice in the control group were administered with vehicle only. Peripheral blood mononuclear cells (PBMC) collected from mice 18-20 hrs after injection of WSPG have significantly higher cytotoxic activity (about 2-fold, P<0.05) against YAC-1, a classical mouse NK-target tumor cell line, as compared to PBMC from vehicle only injected control mice (FIG. 6).

### Example 8

### Anti-Tumor Activity of WSPG in the 4T1 Post-Resection Metastatic Breast Cancer Model

Example 8 shows the effect of WSPG on survival, the number of clonogenic metastatic cells in the lungs, as well as immune suppression and/or activation in a model of 4T1 metastatic breast cancer after surgical removal of the tumor. The 4T1 mouse model is the most clinically relevant and stringent breast cancer model yielding metastatic disease to the lungs, brain and bone, even after primary tumor resection, remarkably similar to human breast cancer (Pulaski et al. Current protocols in immunology 4:20.2.1, 2001; Heppner et al. Breast Cancer Res 2:331-4, 2000).

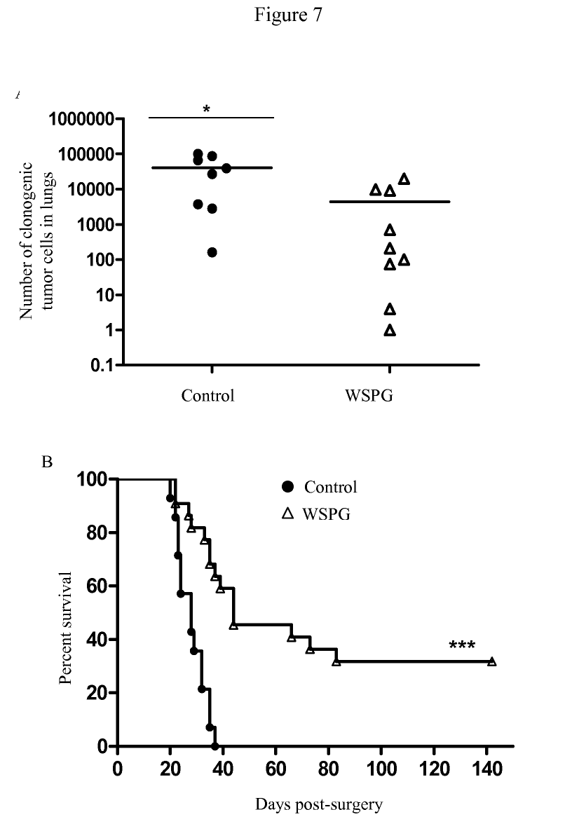

Resection of tumor in 4T1 breast cancer mouse model creates a “window of opportunity” with decreased tumor-associated immune suppression and increased frequency of CD4 and CD8 positive activated T cells, which exists for approximately 10 days (from the fourth to the thirteenth day) after resection (Ghochikyan et al. J Transl Med 12:322, 2014). Administration of WSPG was initiated prior to tumor resection, when tumors were just palpable and was continued during the “window of opportunity” (on 6th and 13th days after tumor resection). One half of the mice were terminated on day 20 post-surgery and clonogenic tumor cells from metastatic disease in the lungs were analyzed, while the other half of mice continued to be injected with WSPG every week until day 69 with survival of mice monitored to day 140. Treatment with WSPG significantly reduced the number of clonogenic tumor cells in lungs of mice detected on day 20 post-resection compared with control animals injected with PBS. Further injections of mice with WSPG resulted in a statistically significant prolongation of survival of mice and complete cure in 31% of the mice (FIG. 7).

The frequencies of activated NK, CD4⁺ and CD8⁺ cells as well as myeloid-derived suppressor cells (MDSC) and Treg suppressor cells were evaluated in spleens of WSPG treated mice on day 20 post-resection. Treatment with WSPG significantly increased frequency of activated NK, CD4⁺ and CD8⁺ cells compared with vehicle injected control mice. The frequency of MDSC was slightly but significantly decreased in the spleens (7.86±1.26% vs 12.43±1.84%, *P<0.05) of mice treated with WSPG relative to control animals. No differences were observed in frequency of Treg cells in spleens of treated and control mice.

### Example 9

### BMDC Pre-Activated with WSPG Reduces Tumor Growth and Number of Metastasis in 4T1 Tumor-Bearing Mice

7×10³ 4 T1 tumor cells were injected subcutaneously into the mammary gland of female BALB/c mice. 5×10⁵ BMDC pre-activated ex vivo with 30 μg WSPG were administered subcutaneously in experimental mice (n=8) three times with weekly intervals. Mice in control group (n=8) were administered with vehicle only. On day 19 tumor size was measured, mice were sacrificed and the number of clonogenic 4T1 tumor cells in lungs were quantified.

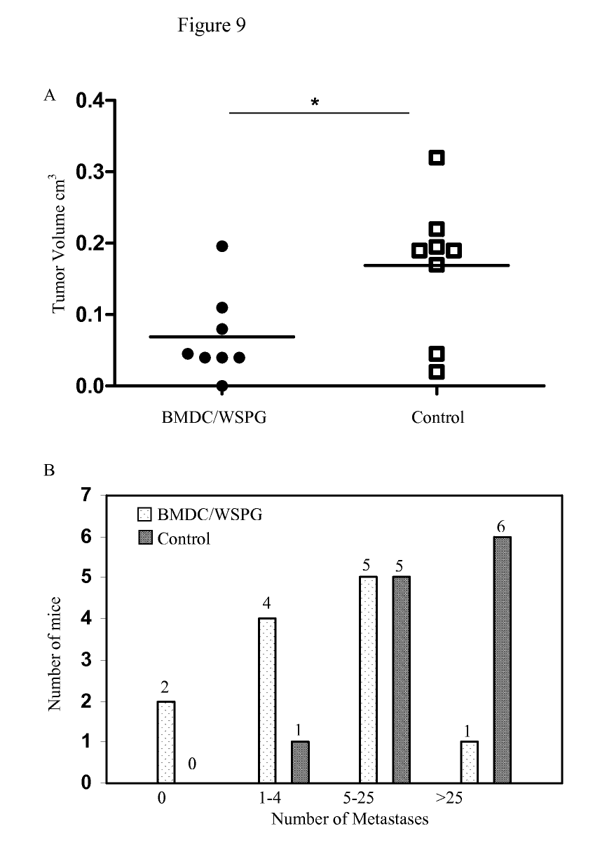

As shown in FIG. 9A mean tumor volume in BMDC/WSPG injected mice was significantly (P<0.05) smaller than in control group.

FIG. 9B shows that 2 mice (out of 12) treated with BMDC/WSPG were free of lung metastases. 6 mice had less than 25 metastases and only one mouse had >25 metastases, while 100% of animals from control group had greater than 5 clonogenic metastatic cells in the lungs. Average numbers of clonogenic metastatic cells were 9.5 and 37.7 in mice vaccinated with BMDC/WSPG and control animals, respectively.

### Example 10

### Mononuclear Cells of Human Peripheral Blood Activated with WSPG Effectively Kill

This example shows the ability of WSPG to activate human antitumor killer cells.

PBMC from healthy donors were incubated (18 hours, 37° C., 5% CO₂) in the absence or presence of WSPG-2 (10 μg/ml). The target K562-cells were labelled by the CellTrace™ Violet. Co-cultures of PBMC and K562 at different effector:target ratios were incubated for 4 hrs at 37° C. and 5% CO₂. Cells were stained with propidium iodide and the percentage of killed target cells was counted on a flow cytometer FACS Aria II. The experiment was repeated using PBMC from three donors.

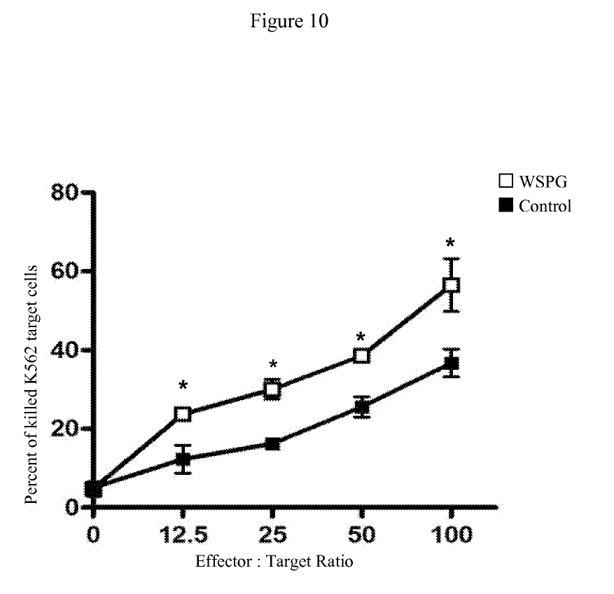

The data in FIG. 10 shows, that WSPG significantly (P<0.05) enhances the cytotoxic activity of human blood mononuclear cells at all tested ratios of effector cells to target cells (E:T).

### Example 11

### WSPG Activates Antitumor Properties of Killer Dendritic Cells of Humans

Dendritic cells were prepared by differentiation of human peripheral blood monocytes from healthy donors as described (Nair et al. Curr Protoc Immunol, Chapter 7:32, 2012). 12.5×10³ moDC cells were co-cultured with 200 4T1-GFP tumor cells for 4 days without or with WSPG-2. The numbers of live 4T1-GFP per well were determined using a flow cytometer BD FACS Aria II as previously described. Data in Table 2 show that the activation of human dendritic cells by WSPG-2 significantly enhances their ability to inhibit the growth of 4T1-GFP cells.

TABLE 2
Inhibition of the growth of tumor cells by human
dendritic cells (MoDC) affected by WSPG.

| | Number of live | | | |
|---|---|---|---|---|
| | 4T1 per well | | | |
| | | Standard | Percent | The |
| Sample | Mean | deviation | inhibition | accuracy |
| 4T1 tumor cells + | 11603 | 717 | | |
| MoDC | | | | |
| 4T1 tumor cells + | 4692 | 86 | 60% | 0.05 * |
| MoDC + WSPG-2, | | | | |
| 10 μg/ml | | | | |

### Example 12

### Injection of Pharmaceutical Composition of WSPG Activates Antitumor Properties of Human Immune Cells

This example shows the ability of WSPG to activate anti-tumor properties of the immune cells directly in the human body after injection of a pharmaceutical-grade WSPG. Studies have been conducted on human volunteers with the pharmaceutical preparation of WSPG designated as “Immunomax” (LLC <<Immafarma” Moscow, P No. 001919/02-2002). “Immunomax” is available for injection in vials. The content of the vial (200 IU) were dissolved in 1 ml of water and administered intravenously.

Blood was collected into BD Vacutainer tubes before the intravenous administration and 1 hour, 3 hours, 7 hours, 24 hours after the intravenous administration for detection of cytokines and analyses of cellular composition of blood. The effect of WSPG was evaluated by characterization of blood lymphocyte populations: detection of activation markers, determination of cytolytic properties (presence of perforin), measuring of cytokines concentration.

Table 3 presents data, showing increasing concentration of immunostimulating cytokines in peripheral blood after intravenous administration of “Immunomax”. After 1 hour the level of TNF-α was increased up to 40 pg/ml and almost returned to the baseline level after 2 hours (Table 3).

TABLE 3
Changes in the content of TNF-α in the blood of volunteers
after the intravenous administration of Immunomax

| Time after | The content of TNF-α in blood, pg/mL | | Significant |
|---|---|---|---|
| injection of | | Standard | differences |
| Immunomax (h) | Mean | deviation | (P) |
| 0 | 3.0 | 2.7 | — |
| 1 | 42 | 42 | 0.05 * |
| 2 | 6.5 | 4.3 | 0.1 |
| 3 | 4.2 | 3.9 | 0.51 |
| 4 | 2.2 | 1.2 | 0.5 |
| 5 | 3.2 | 4.1 | 0.9 |

Tables 4-8 presents the changes in the composition of the immune cells in the peripheral blood of volunteers after intravenous administration of Immunomax.

1-3 hours after the administration of Immunomax, monocytes in the peripheral blood decreased about 2-fold of the baseline, and in 7 hours returned to the original level, and during the next day were increased 3-fold relative to the baseline (Table 4).

TABLE 4
Changes in the content of monocytes in the peripheral blood
of volunteers after intravenous administration of Immunomax

| | | |
|---|---|---|
| Time after | Monocytes in 1 ml of blood | Significant |

| injection | | Standard | differences |
|---|---|---|---|
| Immunomax (h) | Mean | deviation | (P) |
| 0 | 539 | 157 | 0.02 * |
| 1 | 245 | 257 | 0.02 * |
| 3 | 218 | 143 | 0.001 * |
| 7 | 671 | 251 | 0.2 |
| 24 | 1025 | 381 | 0.008 * |

Around the same period of time, changes in the expression of CD49d molecules on the surface of monocytes (Table 5) were observed. CD49d is the integrin (very late antigen-4, VLA-4) that is responsible for the adhesion of monocytes to endothelial cells via vascular cell adhesion molecule-1 (VCAM-1). Expression of VCAM-1 is usually increased at sites of inflammation. Thus, monocytes overexpressing CD49d have greater capacity for adhesion and migration to sites of inflammation, such as in a tumor or metastases. Thus, WSPG may contribute to the body's ability to combat tumor growth by increasing the number, activity and the ability of monocytes to migrate to the center of the tumor.

TABLE 5
Changes in the expression of CD49 on monocytes
in the peripheral blood of volunteers following
intravenous administration of Immunomax.

| | Expression of CD49 | | |
|---|---|---|---|
| | molecules on monocytes, | | |
| Time after | normalized to the initial value | | Significant |
| injection of | | Standard | differences |
| Immunomax (h) | Mean | deviation | (P) |
| 1 | 0.99 | 0.29 | |
| 3 | 0.58 | 0.30 | 0.02 * |
| 7 | 0.57 | 0.20 | 20.00 * |
| 24 | 1.67 | 0.36 | 20.00 * |

Table 6 shows that after 24 hours there is an increase in the expression of HLA-DR molecules on monocytes. HLA-DR molecules on monocytes/macrophages are necessary for antigen presentation to T cells and for the effective development of protective T cell responses against the tumor. The ability of WSPG to increase

TABLE 6
Change in the expression of HLA - DR on monocytes in peripheral
blood of volunteers after intravenous administration of Immunomax

| | Expression of the HLA - | | |
|---|---|---|---|
| | DR molecule on monocytes, | | |
| Time after | normalized to the initial value | | Significant |
| injection of | | Standard | differences |
| Immunomax (h) | Mean | deviation | (P) |
| 1 | 1.2 | 0.4 | 0.2 |
| 3 | 0.5 | 0.1 | 10.00 * |
| 7 | 0.6 | 0.2 | 10.00 * |
| 24 | 1.2 | 0.6 | 0.36 |

the expression level of the antigen-presenting HLA-DR molecules can enhance the specific reaction of the immune system against a tumor.

Injection of “Immunomax” (WSPG) increases the number of cytolytic CD8⁺ T cells containing perforin in the peripheral blood (Table 7). In the first hours after administration of the drug content of CD8⁺ T-cells decreases, but within 1 day, it increased to 1.5 times compared with the level of these cells prior to administration of “Immunomax”. Decrease in the amount of circulating blood cells in the first few hours after the drug administration may indicate their massive migration to the tumor site or to the tissue with inflammation. Later, after 1 day, the drug stimulates the proliferation of cells and the maximum number of CD8⁺ T cells is detected on the third day after the administration of Immunomax. Thus, WSPG promotes specific defense reaction of the immune system against the tumor.

TABLE 7
Changes in the perform content of cytolytic CD8⁺ T cells in
the blood of volunteers after intravenous administration of Immunomax

| | | |
|---|---|---|
| | Content of perform in | |
| | cytolytic CD8⁺ T | |
| Time after | cells in 1 ml of blood | Significant |

| injection of | | Standard | differences |
|---|---|---|---|
| Immunomax (h) | Mean | deviation | (P) |
| 0 | 101 | 70 | |
| 1 | 57 | 46 | <0.05* |
| 3 | 27 | 19 | <0.001* |
| 7 | 30 | 25 | <0.001* |
| 24 | 155 | 101 | 0.2 |

Cytolytic NK cells are generally considered to be a component of the first line of defense against cancer (Lauzon et al. Adv Exp Med Biol 598:1-11, 2007) and play an important role in dealing with the neoplastic cells in the body. As shown in Table 8, even at 1 hour after intravenous injection Immunomax significantly enhanced activation state of NK cells.

TABLE 8
Activation of NK cells (upregulation of CD69) in volunteers
after intravenous administration of Immunomax

| | The content of activated CD 69+ NK | | |
|---|---|---|---|
| | cells in the blood, normalized to the | | |
| Time after | initial value (Before administration) | | Significant |
| injection of | | Standard | differences |
| Immunomax (h) | Mean | deviation | (P) |
| 1 | 2.10 | 0.13 | <0.05 * |
| 3 | 5.87 | 0.32 | <0.05 * |
| 7 | 5.23 | 0.64 | <0.05 * |
| 24 | 1.29 | 0.85 | . |

Overall, the data presented in Example 12 indicate that Immunomax activates protective immune cells (monocytes, CD8⁺ T cells and NK cells) and directs them to the pathologically altered tissues. This effect of the drug may provide the increased protection against cancer.

We claim:

1. A method of new use of the compound water-soluble acidic peptidoglycan from sprouts of the plant Solanum tuberosum (WSPG) for treating cancerous tumors and preventing, treating, inhibiting and/or controlling the formation or establishment of metastases at one or more sites distinct from primary tumor or cancer.

2. The method according to claim 1 wherein a pharmaceutically effective amount of the compound WSPG and a pharmaceutically acceptable carrier, excipient and/or diluent are administered to a patient in need thereof.

3. The method according to claim 1 wherein a pharmaceutically effective amount of mammalian immune cells treated with compound WSPG or with similarly acting other TLR4-agonist in vitro/ex vivo are administered to a patient in need thereof.

4. The method according to claim 1 wherein said WSPG is a compound according to the patent RU 2195308.

5. The method according to claim 1 wherein said WSPG is a compound according to the patent application RU 2013151824.

6. The method according to claim 1 wherein said WSPG is a pharmacological compound registered in Russian Federation under No. 001919/02-2002

7. The method according to claim 1 wherein said compound WSPG is an immunostimulator.

8. The method according to claim 1 wherein said compound WSPG activates dendritic cells and macrophages transforming them into killer cells and/or tumor cell growth inhibitors.

9. The method according to claim 1 wherein said compound WSPG activates NK cells, increasing their antitumor activity.

10. The method according to claim 3, wherein said mammalian cells are autologous peripheral blood mononuclear cells or dendritic cells or macrophages.

11. The method according to claim 3, wherein said mammalian cells activated with WSPG and administered to a patient in need thereof inhibit the growth of tumor and the spread of metastases.

12. The method of claim 1, for use in treating cancer selected from the group consisting of melanoma, colon, breast, prostate, bladder, lung, kidney, liver, stomach, pancreas, ovary, uterus, head and neck, brain, bone marrow and a hematological malignancy, as well as sarcoma, glandular tissue, skin tumor, mucosal and other epithelia.

13. An immune cell activated with compound WSPG endowed with tumoricidal activity, said cell selected from a group of cells comprising of: a) T cells; b) monocytes; c) dendritic cells; d) macrophages; e) NK cells; f) peripheral blood mononuclear cells; g) NKT cells; h) lymphokine activated killers; i) NK-92 cells; j) DC-CIK; k) combination of dendritic cells and NK cells; l) dendritic cells and T cells; and m) CD8 T cells cultured with antigen presenting cells.

14. The immune cell of claim 13, wherein said immune cell is pretreated with a source of tumor antigen.

15. The immune cell of claim 13, wherein said tumoricidal activity is determined by ability to reduce viability of a malignant cell.

16. A pharmaceutical kit for treatment of cancer comprising an antibody derived therapeutic whose activity is mediated in part by antibody dependent cytotoxicity (ADCC) and a therapeutically sufficient amount of WSPG capable of augmenting activity in vivo of cells mediating ADCC.

17. The pharmaceutical kit of claim 16, wherein said antibody derived therapeutic whose activity is mediated in part by ADCC is selected from a group of antibodies comprising of: a) Herceptin; b) rituximab; and c) cetuximab.

18. A pharmaceutical kit for treatment of cancer comprising a checkpoint inhibitor and a therapeutically sufficient amount of WSPG capable of augmenting in vivo activation of innate immune cells.

19. The pharmaceutical kit of claim 18, wherein said checkpoint inhibitor is an inhibitor of immune associated proteins selected from the group comprising of: a) PD-1; b) PD-1 ligand; c) CTLA-4; d) TIM-1; and d) LAG-3.

20. The method of treating cancer with immune cell of claim 13, wherein said immune cells are autologous or allogeneic.

21. A method of treating cancer comprising administration of a therapeutic amount of WSPG and an intervention selected from a group of interventions comprising of: a) chemotherapy; b) radiotherapy; c) hormone therapy; and d) anti-angiogenic therapy.
